(19) United States
(12) Patent Application Publication
(10) Pub. No.: US 2022/0326155 A1
(43) Pub. Date: Oct. 13, 2022

(54) TIME-RESOLVED FLUORESCENCE AND CHROMOGENIC DUAL-SIGNAL TEST STRIP FOR ESTROGEN AND ITS PREPARATION METHOD AND APPLICATION

(71) Applicant: JIANGNAN UNIVERSITY, Wuxi, Jiangsu (CN)

(72) Inventors: Yi ZHANG, Wuxi, Jiangsu (CN); Yongxi HE, Wuxi, Jiangsu (CN); Wenlong WANG, Wuxi, Jiangsu (CN); Lingling WANG, Wuxi, Jiangsu (CN); Xiuping YAN, Wuxi, Jiangsu (CN)

(21) Appl. No.: 17/623,244

(22) PCT Filed: Nov. 11, 2021
(86) PCT No.: PCT/CN2021/130133

§ 371 Date: Dec. 28, 2021

(30) Foreign Application Priority Data
Mar. 15, 2021 (CN) 202110274596.4

(51) Int. Cl. G01N 21/64; G01N 33/533; G01N 33/577; G01N 33/58

(52) U.S. Cl. CPC G01N 21/6408; G01N 33/533; G01N 33/577; G01N 33/582; G01N 33/587

(57) ABSTRACT

A time-resolved fluorescence and chromogenic dual-signal test strip for estrogen is based on the principle of immune recognition and fluorescence resonance energy transfer. In time-resolved fluorescence mode, estrogen-BSA-persistent luminescence particle complex is the fluorescence donor, and colloidal gold modified with estrogen monoclonal antibody is the fluorescence receptor, which is also chromogenic signal unit in the chromogenic mode. The photos of strips in both modes are obtained with smart phones. The time-resolved fluorescence intensity of the test strip test zone is positively correlated with estrogen content, and the chromogenic intensity is negatively correlated with estrogen content. The competitive time-resolved fluorescence and chromogenic dual-signal immunochromatographic test strips can accurately and quickly detect estrogen and estrogen-like compounds.

### BACKGROUND OF THE INVENTION

### 1. Technical Field

The present invention relates to a time-resolved fluorescence and chromogenic dual-signal immunochromatographic test strip for estrogen and its preparation method and application, which belongs to the field of analysis and detection.

### 2. Background Art

Estrogen (E) is a substance that promotes the development of secondary sexual characteristics and the maturation of sexual organs in female animals. It is secreted by the ovaries and placenta of female animals. Estrogen has a wide range of important physiological effects. It not only promotes and maintains the physiological effects of female reproductive organs and secondary sexual characteristics, but also has obvious effects on endocrine, cardiovascular, metabolic systems, bone growth and maturation, skin, etc. Natural estrogen mainly includes estradiol E2, estrone E1, estriol E3. Currently, clinical estrogen drugs are commonly artificially synthesized derivatives of estradiol, such as diethylstilbestrol and ethinyl estradiol. 17β-estradiol is the most active natural estrogen among all endocrine disruptors and is often used as an endocrine regulation drug for menopausal women. However, when estrogen accumulates in the human body through drinking water and food and exceeds the safety threshold, it will damage human health, disrupt the balance of the body, and even endanger future generations.

At present, the detection methods of estrogen mainly include chromatography, electrochemistry, and surface-enhanced Raman spectroscopy (SERS). The chromatographic method has high sensitivity and good stability, but it cannot be applied to on-site detection. The electrochemical method and SERS method are fast and sensitive, which can be used for on-site detection, but lack of stability and reproducibility.

Test strip, a paper-based rapid detection technology, is low-cost, easy to operate, and can realize on-site detection. It has been widely used in clinical diagnosis, food safety, environmental monitoring and other fields. Colloidal gold is often used as a chromogenic probe for test strips because its chromogenic results are visible to the naked eye, but its sensitivity is low, and the reading results are subjective. In order to improve the sensitivity of the test strip, gold nanoflowers/gold nanorods or secondary labeling of colloidal gold can be used, or fluorescent lateral flow analysis test strips can be developed, using small molecule organic dyes, quantum dots, up-conversion nanoparticles, etc. as signal probes. However, the above-mentioned fluorescent lateral flow analysis test strip has disadvantages, such as the interference of the fluorescent background from the test strip or the need for a high-energy laser light source.

### SUMMARY OF THE INVENTION

In view of current technical defects and detection needs, the purpose of the present invention is to provide a new, specific and rapid time-resolved fluorescence (TRF) and chromogenic dual-signal immunochromatographic test strip for estrogen determination, and its preparation method and application.

The present invention is based on the principle of immune recognition and fluorescence resonance energy transfer, using persistent luminescent particles (PLPs) coupled with estrogen-bovine serum albumin (E-BSA) as fluorescent donors and estrogen monoclonal antibody-modified colloidal gold (CG-mAb) nanoparticles as fluorescent acceptors, and the competitive TRF and chromogenic dual-signal test strip for estrogen has been established. This method uses a low-power ultraviolet lamp as the excitation light source, and uses the smart phone continuous shooting function to collect the fluorescent images of the test strip's test zone to realize the TRF mode, which effectively removes the fluorescent background interference from the test strip and improves the signal-to-noise ratio. The large persistent luminescence particles with excellent afterglow are used as the fluorescent signal source. The estrogen competitor E-BSA coupled PLPs are fixed at the test zone due to their non-migration feature on the nitrocellulose membrane, which ingeniously solves the contradiction between the conventional test strip's nano-level limitation on the size of the signal material and the short-term afterglow of small-sized persistent luminescence nanoparticles. TRF and chromogenic dual-signal detection combines naked eye quick screening with precise quantification, and meanwhile increase the credibility of the results. This method can be used for rapid and sensitive detection of estrogen.

The purpose of the present invention has been achieved through the following technical proposals:

The first object of the present invention is to provide an estrogen time-resolved fluorescence and chromogenic dual-signal test strip. The dual-signal test strip consists of a sample pad, a nitrocellulose (NC) membrane and an absorbent pad, all of which were affixed on a PVC bottom plate with both ends of the NC membrane located in the lower overlapping layer. The NC membrane is used to separate and detect analytes from other substances in the sample. The sample pad is used for sample loading. The absorbent pad is used to absorb excess liquid. The PVC bottom plate provides physical support for the test strip;

The NC membrane includes a test zone, namely T zone, and a quality control zone, namely C zone;

The test zone is fixed with a complex of estrogen-bovine serum albumin-persistent luminescent particles (E-BSA-PLPs). The estrogen refers to chemicals having phenolic hydroxyl groups and an estrogen effect, including estradiol (E2), estriol (E3), estrone (E1), bisphenol A (BPA), diethylstilbestrol, ethinyl estradiol, etc. The estrogen effect refers to the physiological effect similar to estrogen that can be produced on the endocrine system by acting with estrogen receptor, also called estrogen-like effect.

A secondary antibody is fixed on the quality control zone.

In one embodiment, the secondary antibodies fixed in the quality control zone include but are not limited to goat anti-mouse secondary antibodies, rabbit anti-mouse secondary antibodies, goat anti-rabbit secondary antibodies, and donkey anti-rabbit secondary antibodies.

Another object of the present invention is to provide a method for preparing estrogen time-resolved fluorescence and chromogenic dual-signal test strips. The preparation steps are as follows:

(i) Synthesize estrogen-bovine serum albumin conjugate (E-BSA);

(ii) Carboxyl modification PLPs;

(iii) Prepare the complex of E-BSA with PLPs (E-BSA-PLPs), and store them in phosphate buffered saline solution (PBS) for later use;

(iv) Construct the test strips: dropping E-BSA-PLPs solutions in T zone, dropping secondary antibody in C zone, and storing the strips in vacuum bags after drying.

In one embodiment, in the step (iii), the active ester method is used for biological coupling, and the specific method is: ultrasonically disperse the carboxyl modified PLPs into PBS, and then add 1-ethyl-3-(3-dimethylaminopropyl) carbodiimide hydrochloride (EDCHC1) and N-hydroxysuccinimide (NETS) with stirring at room temperature, and then centrifuge to remove the supernatant and wash the precipitate with PBS, and then add E-BSA and continue stirring overnight, and then collect the precipitate coupling product by centrifugation, wash with PBS and re-dissolve in PBS for later use.

In one embodiment, in the step (iv), the T zone is dripped with a 5-20 mM PBS solution containing 0.5-5 mg/mL E-BSA-PLPs, and the C zone is dripped with a 5-20 mM PBS solution containing 0.05-0.5 mg/mL secondary antibody.

In one embodiment, in the step (ii), E-BSA is synthesized by the estrogen carboxymethyl ether (E-CME) and BSA with the active ester method, and in the step (iii), the PLPs and carboxyl-ethyl-silanetriol sodium salt (CES) are subjected to a condensation reaction to obtain carboxyl-modified PLPs.

Another object of the present invention is to provide a detection device for detecting estrogen. This device comprises a test strip and CG-mAb. And the test strip is the above-mentioned time-resolved fluorescence and chromogenic dual-signal test strip for estrogen. In the time-resolved fluorescence mode, E-BSA-PLPs complex on the test strip is fluorescent donor and CG-mAb nanoparticle is fluorescent acceptor, whereas in the chromogenic mode, CG-mAb nanoparticle is the chromogenic signal unit.

In one embodiment, the method for preparing CG-mAb is as follows: prepare estrogen monoclonal antibody modified colloidal gold to obtain CG-mAb. The monoclonal antibodies used to modify colloidal gold are from, but are not limited to, mice, rats, and rabbits. The CG-mAb is stored in a closed container including a centrifuge tube in the state of a solution or a freeze-dried powder. Correspondingly, the fixed secondary antibody in the quality control zone is a secondary antibody directed against the primary antibody source.

In one embodiment, the C zone is used as a reference to verify the validity of the test strip results, and it always shows red and no fluorescence. The time-resolved fluorescence intensity of the T zone is positively correlated with the estrogen content in the sample, and the chromogenic intensity is negatively correlated with the estrogen content in the sample. Specifically:

When the sample does not contain estrogen, the T zone will be red, and the fluorescence will be quenched;

When the sample contains estrogen, the T zone will appear light red or even disappear, and the fluorescence will increase.

In one embodiment, the application method of the detection device is as follows: put the sample to be tested and the CG-mAb in the running buffer and mix for 3-10 min, put the test strip into the mixture and read the chromogenic result after 15-30 minutes; the running buffer is 8-15 mmol/L, of which pH is 6.5-8.0, containing 5-15% sucrose, 6-10% BSA, 0.15-0.30% Tween-20, and the buffer includes but is not limited to phosphate buffer, borate buffer, carbonate buffer.

After the chromogenic detection is completed, place the test strip under the ultraviolet light to excite, use the fluorescent image acquisition device to obtain the fluorescent images before and after the ultraviolet light source is turned off, and read the fluorescent results.

In another embodiment, the fluorescence image acquisition device includes a smart phone, and the smart phone obtains fluorescence images before and after the ultraviolet light source is turned off in a continuous shooting mode, and obtains time-resolved fluorescence photos.

The principle of the time-resolved fluorescence and chromogenic dual-signal immunochromatographic test strip for fast detection of estrogen is as follows, using estradiol as an example: The C zone is used as a reference to verify the validity of the test paper results, always showing red without fluorescence. The sample solution and CG-mAb are premixed and then move towards the absorbent paper under capillary action. When there is no estradiol in the sample, the CG-mAb is captured by the E2-BSA-PLPs in the T zone, and the T zone will be visible red to the naked eye, whereas FRET will occur between E2-BSA-PLPs and CG-mAb, quenching the fluorescence of the PLPs in the T zone. Excess CG-mAb will be captured by the secondary antibody in the C zone and the C zone will always show red without fluorescence. When the sample contains estradiol, estradiol will combine with part of the CG-mAb to reduce the total amount of CG-mAb captured in the T zone. And the T-zone can be seen to be light red by the naked eye or even disappears, whereas the FRET between E2-BSA-PLPs and CG-mAb is reduced or even disappears, and the fluorescence of the PLPs is restored. As the concentration of estradiol in the sample increases, the amount of CG-mAb captured in the T zone decreases, thus the brightness of the red color in the T zone is inversely proportional to the concentration of the analyte, whereas the fluorescence intensity of the PLPs in the T zone is proportional to the concentration of the analyte.

The invention has the following advantages:

The time-resolved fluorescence and chromogenic dual-signal immunochromatographic test strip for estrogen is based on the principle of fluorescence resonance energy transfer. PLPs with excellent afterglow and micron size are used as T-zone fixed fluorescent probes, being excited by low-power ultraviolet combining with smart phone shooting to achieve TRF mode fluorescence collection. The fluorescence background interference of the test strip is eliminated effectively and the signal-to-noise ratio is greatly improved. Compared with colloidal gold colorimetry, the detection limit of this method is two orders of magnitude lower (0.1 mg/mL) and the specificity is also better. Compared with similar fluorescent detection test strips, this method has less background pollution and faster detection speed. It is suitable for on-site screening.

### BRIEF DESCRIPTION OF THE DRAWINGS

FIG. 1 is a schematic diagram of the time-resolved fluorescence and chromogenic dual-signal immunochromatographic test strip for estrogen.

FIG. 2 is the response of the test strip to the spiked drinking water samples: chromogenic mode (A), TRF mode (B).

FIG. 3 is the standard curve of the test strip: chromogenic mode (A), TRF mode (B).

FIG. 4 is the response of the test strip to different concentrations of estradiol: chromogenic mode (A), TRF mode (B).

### DESCRIPTION OF PREFERRED EMBODIMENTS

The detailed implementation of the invention is further described as follows. The following embodiments are used to illustrate the invention, but not to limit the scope of the invention. Technicians in this field can clearly understand the characteristics and efficacy of the invention from the contents explained in this specification, and the invention can also be implemented or applied in other specific ways. Except for special instructions, the materials and reagents etc. used in the invention can be obtained from commercial sources.

The following examples take estradiol as an example of estrogen to fully describe the preparation of the entire test strip and the use of the detection device.

### EXAMPLE 1

The construction of TRF and chromogenic dual-signal immunochromatographic test strip for estrogen and its application in a drinking water sample are as follows:

1. Preparation of Test Strip Materials

1.1. Preparation of Colloidal Gold (CG) and CG Modified by Monoclonal Antibody Against Estradiol (CG-mAb)

CG is prepared by sodium citrate reduction method, and CG-mAb is prepared by coupling CG with estradiol monoclonal antibody by adsorption, and the product after centrifugation (30 min, 4° C., 10000 r/min) is dissolved in 0.2 mL buffer (20 mmol/L borate buffer containing 5% sucrose, 1% BSA, pH 8.2) and stored for later use.

1.2. Synthesis of Estradiol Carboxymethyl Ether (E2-CME) and E2-BSA Conjugate

Dissolving 100 mg of estradiol and 0.5 g of KCl in 6 mL of dimethyl sulfoxide (DMSO), and ultrasonicating for 5 min. Under stirring, 100 mg of bromoacetic acid is added. After 2 h of reaction, 50 mL of ice water is added to terminate the reaction. Extracted the unreacted estradiol with ethyl acetate, add 2 mol/L hydrochloric acid dropwise to the aqueous phase to acidify the solution until the white precipitate appeared. After centrifugation, the supernatant is removed, and the precipitate is washed with ultrapure water to pH≈7 and then vacuum freeze-dried to obtain E2-CME.

E2-BSA is synthesized by the active ester method. Specifically, dissolve 3.3 mg of E2-CME in 0.5 mL DMSO, adding 6 mg NHS and 7 mg EDCHCl, and stir for 12 h to react. Dissolve 20 mg of BSA in 4 mL of carbonate buffer solution (CBS) whose concentration is 50 mM and pH is 9.6, and add the activated E2-CME solution slowly to the CBS solution and continue to stir for 12 h. After the reaction, transfer the mixture to a dialysis bag and put in 1 L of PBS for dialysis whose concentration is 10 mM and pH is 7.4. Change the pressure fluid every 12 h for 3 consecutive days. After the dialysis, the E2-BSA conjugate is obtained and frozen for further use.

1.3. Modification of PLPs

Dissolve 30 mg of PLPs in 5 mL of absolute ethanol under ultrasonication in a 10 mL centrifuge tube. Add 20 mL of absolute ethanol, 5 mL of water, and 0.13 mL of tetraethylorthosilicate (TEOS) to a 50 mL round-bottom flask. Under stirring, put the PLPs-ethanol solution into the round-bottom flask quickly and sonicate for 10 minutes to thoroughly mix the solution. Add 0.5 mL ammonia (28-30%) to the mixture, sonicating for 30 min and then stir the mixture for 7.5 h. After the reaction, Centrifuge and wash 3 times with absolute ethanol under the centrifugal conditions of 10000 r/min, 10 min. Dry the product under vacuum to get silica-encapsulated PLPs.

Disperse 25 mg of silica-encapsulated PLPs into 25 mL of water ultrasonically, adding 100 μL of CES under electromagnetic stirring, and stir it at room temperature for 24 h. After centrifugation, wash the precipitate using ultrapure water and absolute ethanol twice each, and dry it in vacuum at room temperature to obtain carboxyl-modified PLPs.

1.4. Preparation of E2-BSA-PLPs Complex

The E2-BSA-PLPs complex is prepared by coupling E2-BSA and carboxyl-modified PLPs by the active ester method. The specific method is: dispersing the carboxyl-modified PLPs into PBS ultrasonically, adding 6 mg of EDCHCl and 15 mg of NHS, stirring at room temperature and centrifuging to remove the supernatant, adding 1 mL of E2-BSA at a concentration of 1 mg/mL, stirring overnight, centrifuging and collecting the precipitate, washing the precipitate with PBS and dissolving the product E2-BSA-PLPs complex in PBS for later use. The product is stored in PBS solution at 2 mg/mL (calculated as the concentration of PLPs) for later use.

1.5. Preparation of Test Zone (T Zone) Solution: E2-BSA-PLPs is Diluted to 2 mg/mL with 10 mM PBS solution.

1.6. Preparation of Quality Control Zone (C Zone) Solution: the Goat Anti-Mouse Secondary Antibody is Diluted to 0.1 mg/mL with 10 mM PBS Solution.

2. Preparation of Test Strips

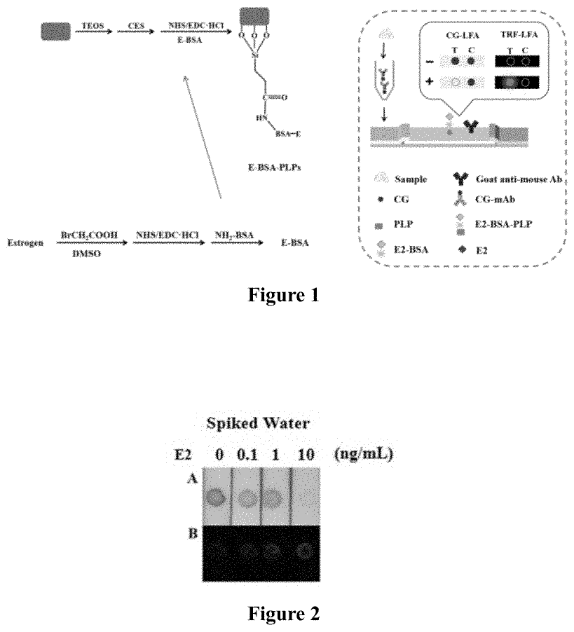

According to FIG. 1, the NC film was pasted in the middle of the PVC baseplate, the sample pad and the absorbent pad are overlapped on the left and right ends of the NC film respectively, and then they are cut into strips of 3 mm width, obtaining blank test strips. 1.0 μL T zone solution and 0.5 μL C zone solution are dropwise added in the T zone and C zone of the test strips respectively. The test strips are dried at 37° C. for 30 min, and stored in a vacuum bag for later use.

3. Drawing Working Curve

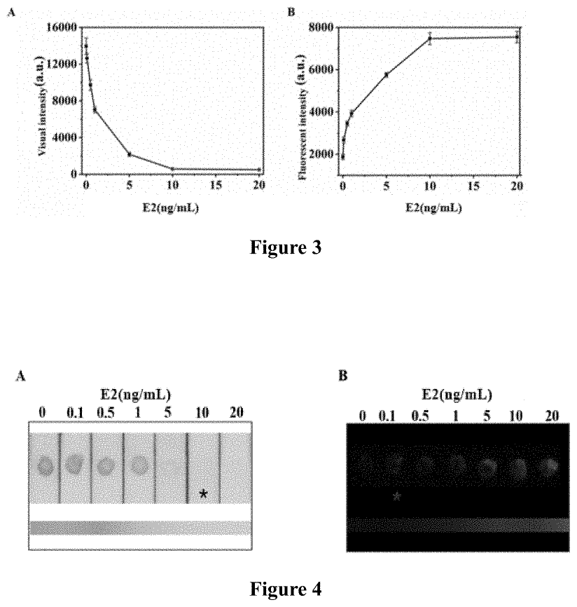

10 mL of 1 mg/mL E2 standard stock solution is prepared with methanol and it was diluted with 10 mM PBS to concentrations of 0 mg/mL, 0.1 mg/mL, 0.5 mg/mL, 1 mg/mL, 5 mg/mL, 10 mg/mL, 20 mg/mL of E2, and this series is the standard working solutions. 80 μL of the test solution, 20 μL CG-mAb and 10 μL running buffer (10 mmol/L phosphate buffer, 10% sucrose, 8% BSA, 0.25% Tween-20, pH 7.4) are mixed in a centrifuge tube for 5 min. A test strip is inserted into the centrifuge tube, and the result is analyzed after 25 minute. The result of chromogenic mode is shown in FIG. 4A, and the corresponding working curve is shown in FIG. 3A. The result of TRF mode is shown in FIG. 4B, and the corresponding working curve is shown in FIG. 3B. According to FIG. 4A, in the chromogenic mode, the T zone of the negative and low-concentration positive shows a clear purple-red color, whereas the color of the T zone becomes lighter when the E2 concentration increases to 5 ng/mL, and the color of the T zone disappears at 10 ng/mL. This concentration at which T zone disappears is the detection limit of the chromogenic method; in TRF mode, there is no obvious fluorescence in the negative T zone. When the E2 concentration is 0.1 ng/mL, the T zone emits weak fluorescence (FIG. 4B), and this concentration is the detection limit of the TRF method. Therefore, the detection limit of the constructed test strip can reach 0.1 mg/mL in TRF mode.

4. Sample Pretreatment

First, the drinking water sample is filtered and detected with high performance liquid chromatography-mass spectrometry (HPLC-MS). It is estradiol-negative. Add 800 μL of E2 standard solutions with concentrations of 0 mg/mL, 0.10 mg/mL, 1 mg/mL, and 10 mg/mL, respectively, into 1 mL drinking water samples to get a series of spiked samples.

5. Sample Detection

Mix 80 μL of the sample solution with 20 μL of CG-mAb and 10 μL of running buffer (10 mmol/L phosphate buffer, 10% sucrose, 8% BSA, 0.25% Tween-20, pH 7.4) in a centrifuge tube for 5 minute, and then inserted the test strip into the centrifuge tube, and read the chromogenic result after 25 minutes. After the chromogenic detection is completed, placed the test strip under an ultraviolet lamp to excite the fluorescence of PLPs, use a smartphone to obtain the fluorescence images before and after the ultraviolet light source is turned off, and analyze the image data. The results are shown in FIG. 2, indicating the test strip can detect at least 0.1 ng/mL of E2. Moreover, the analysis can be completed in about 30 minutes.

### EXAMPLE 2

The construction of TRF and chromogenic dual-signal immunochromatographic test strip for estrogen and its application in a milk sample are as follows:

1. Preparation of Test Strip Materials

Same as Example 1

2. Preparation of Test Strips

Same as Example 1

3. Drawing Working Curve

Same as Example 1

4. Sample Pretreatment

First, the milk sample is detected with HPLC-MS and is found to be estradiol-negative. The milk sample is centrifuged by 8000 r/min at 10° C. for 10 min, and then the upper milk fat is discarded to obtain skim milk. Diluted the skim milk with deionized water at a volume ratio of 1:20, filtered, measured 4 portions, 1 mL each, and put them into four 10 mL centrifuge tubes, respectively, as the test solution for use.

5. Sample Detection

Mix 80 μL of the sample solution with 20 μL of CG-mAb and 10 μL of running buffer (10 mmol/L phosphate buffer, 10% sucrose, 8% BSA, 0.25% Tween-20, pH 7.4) in a centrifuge tube for 5 minute, and then insert the test strip into the centrifuge tube, and read the chromogenic result after 25 minutes. After the chromogenic detection was completed, place the test strip under an ultraviolet lamp to excite the fluorescence of PLPs, use a smartphone to obtain the fluorescence images before and after the ultraviolet light source is turned off, and analyze the image data.

The concentration of E2 in the milk sample is found to be lower than the limit of quantification and the limit of detection.

### EXAMPLE 3

The construction of TRF and chromogenic dual-signal immunochromatographic test strip for estrogen and its application in a pork sample of are as follows:

1. Preparation of Test Strip Materials

Same as Example 1

2. Preparation of Test Strips

Same as Example 1

3. Drawing Working Curve

Same as Example 1

4. Sample Pretreatment

First, the pork sample is detected with HPLC-MS and was found to be estradiol-negative. Weigh 100 g of the pork without bones and skin, crush and then homogenize the pork sample. Weigh 4 portions of homogenized pork sample, 1 g each, into centrifuge tubes, add 3 ml of ethyl acetate, 0.1 g of anhydrous calcium oxide, and shake for 2 min, centrifuge at 4000 r/min for 10 min, transfer the supernatant to another test tube, dry with nitrogen flow, add 1 mL of n-butane to dissolve the residue, added 0.5 mL of PBS-methanol solution (v:v=3:2, pH=7.0), shake for 2 min, then centrifuge at 4000 r/min for 10 min, remove n-butane in the upper layer, and take the solution in the lower layer as the test solution.

5. Sample Detection

Mix 80 μL of the sample solution with 20 μL of CG-mAb and 10 μL of running buffer (10 mmol/L phosphate buffer, 10% sucrose, 8% BSA, 0.25% Tween-20, pH 7.4) in a centrifuge tube for 5 minute, and then insert the test strip into the centrifuge tube, and read the chromogenic result after 25 minute. After the chromogenic detection is completed, place the test strip under an ultraviolet lamp to excite the fluorescence of PLPs, use a smartphone to obtain the fluorescence images before and after the ultraviolet light source is turned off, and analyze the image data. The concentration of E2 in the milk sample is0 found to be lower than the limit of quantification and the limit of detection.

Example 1 shows that the test strip can detect spiked E2 at 0.1 ng/mL and above. Moreover, the analysis can be completed only in 30 minutes and the test strips and related testing materials are easy to store. The test strips can be used in a wide range of samples, such as Examples 2 and 3.

In summary, the test strip and the corresponding detection device of the present invention have good estradiol detection performance and applicability. Similarly, it can also be extended to estrogens and estrogen-like compounds, which has the same phenolic hydroxyl structure as estradiol and has an estrogenic effect, such as estriol, estrone, bisphenol A, diethylstilbestrol, ethinyl estradiol, etc. For each estrogen analyte, replace the E2's antibody with the corresponding primary antibody. The phenolic hydroxyl group in the estrogen structure can be derived to a carboxyl group through a series of chemical reactions, and then the estrogen can be coupled to BSA through this carboxyl group.

The foregoing is only the preferred embodiments of the present invention. It should be pointed out that for the common technical personnel in this technical field, without departing from the principle of the present invention, several improvements and modifications can be made, and these improvements and modifications are also should be regarded as the protection scope of the present invention.

1. A time-resolved fluorescence and chromogenic dual-signal immunochromatographic test strip for estrogen comprises a sample pad, a nitrocellulose membrane that is NC membrane with two ends, and an absorbent pad on the PVC bottom plate in order along a horizontal direction, and the two ends of the NC membrane are located in a lower layer where the NC film overlaps with the sample pad and absorption pad;
the NC membrane comprises a test zone, namely T zone, and a quality control zone, namely C zone;
the T zone is fixed with estrogen-bovine serum albumin-persistent luminescent particles (E-BSA-PLPs), and the estrogen is an estrogen compound or an estrogen-like compound with phenolic hydroxyl groups and an estrogen effect, including estradiol, estriol, estrone, bisphenol A, diethylstilbestrol, and ethinyl estradiol;
secondary antibody is fixed on the quality control zone.

2. The time-resolved fluorescence and chromogenic dual-signal immunochromatographic test strip for estrogen of claim 1 wherein the secondary antibody fixed in the C zone includes, but is not limited to, goat anti-mouse antibody, rabbit anti-mouse antibody, goat anti-rabbit antibody and donkey anti-rabbit antibody.

3. The method of preparing the time-resolved fluorescence and chromogenic dual-signal immunochromatographic test strip for estrogen of claim 1 comprising:
(i) synthesizing estrogen-bovine serum albumin conjugate E-BSA;
(ii) carboxyl modification of PLPs;
(iii) preparing the complex of E-BSA and persistent luminescent particles E-BSA-PLPs;
(iv) constructing the test strips: dropping E-BSA-PLPs in T zone, dropping the secondary antibody against the source of the primary antibody in C zone, and storing the strips in vacuum bags after drying.

4. The method of preparing the time-resolved fluorescence and chromogenic dual-signal immunochromatographic test strip for estrogen of claim 3 wherein the active ester method for biological coupling is used in the step (iii), specifically, ultrasonically disperse the carboxyl modified PLPs into PBS, and then add 1-ethyl-3-(3-dimethylaminopropyl) carbodiimide hydrochloride and NHS with stirring at room temperature, and then centrifuge to remove the supernatant and wash the precipitate with PBS, and then add E-BSA and continue stirring overnight, and then collect the precipitate coupling product by centrifugation, wash with PBS and re-dissolve in PBS for later use.

5. The method of preparing the time-resolved fluorescence and chromogenic dual-signal immunochromatographic test strip for estrogen of claim 3 wherein in the step (iv), the T zone is dripped with a 5-20 mM PBS solution containing 0.5-5 mg/mL E-BSA-PLPs, and the C zone is dripped with a 5-20 mM PBS solution containing 0.05-0.5 mg/mL secondary antibody.

6. The method of preparing the time-resolved fluorescence and chromogenicdual-signal immunochromatographic test strip for estrogen of claim 3 wherein in the step (ii), E-BSA is synthesized by the estrogen carboxymethyl ether (E-CME) and BSA with the active ester method, and in the step (iii), the PLPs and carboxyl-ethyl-silanetriol sodium salt (CES) are subjected to a condensation reaction to obtain carboxyl-modified PLPs.

7. A detection device for detecting estrogen comprising a test strip and an estrogen monoclonal antibody modified colloidal gold, that is, CG-mAb, and the test strip is the time-resolved fluorescence and chromogenic dual-signal immunochromatographic test strip for estrogen of claim 1 or 2, or test strips prepared by the method of any one of claims 3-6, and the detection device uses E-BSA-PLPs complex on the test strip as fluorescent donors and CG-mAb as fluorescent acceptors.

8. The detection device for estrogen of claim 7 wherein the C zone is used as a reference for verifying the validity of the test strip, and it always shows red and no fluorescence; the time-resolved fluorescence intensity of the T zone is positively correlated with the estrogen content in the sample, and the chromogenic intensity is negatively correlated with the estrogen content in the sample, specifically,
when the sample does not contain estrogen, the T zone becomes red, and the fluorescence is quenched;
when the sample contains estrogen, the T zone appears light red or even colorless, and the fluorescence shows up.

9. The detection device for estrogen of claim 7 wherein the application method of the detection device is as follows: put the sample to be tested and the CG-mAb in the running buffer and mix for 3-10 min, put the test strip into the mixture and read the chromogenic result in 15-30 min; the running buffer is 8-15 mmol/L of which pH is 6.5-8.0, containing 5-15% sucrose, 6-10% BSA, 0.15-0.30% Tween-20, and the buffer comprises phosphate buffer, borate buffer, and carbonate buffer;
after the chromogenic detection is completed, place the test strip under the ultraviolet light to excite, use the fluorescent image acquisition device to obtain the fluorescent images before and after the ultraviolet light source is turned off, and read the fluorescent results.

10. The detection device for estrogen of claim 9 wherein the fluorescence image acquisition device comprises a smart phone, and the smart phone obtains fluorescence images before and after the ultraviolet light source is turned off in a continuous shooting mode and obtains time-resolved fluorescence photos.
